(19) United States
(12) Patent Application Publication
(10) Pub. No.: US 2025/0325814 A1
(43) Pub. Date: Oct. 23, 2025

(54) SYSTEMS AND METHODS FOR DETECTING LEAD MOVEMENT

(71) Applicant: Medtronic, Inc., Minneapolis, MN (US)

(72) Inventor: Joshua James Nedrud, Roseville, MN (US)

(21) Appl. No.: 18/862,663

(22) PCT Filed: Apr. 3, 2023
(86) PCT No.: PCT/US2023/065264

§ 371 Date: Nov. 4, 2024

Related U.S. Application Data
(60) Provisional application No. 63/338,280, filed on May 4, 2022.

(51) Int. Cl. A61N 1/36; A61N 1/05

(52) U.S. Cl. CPC A61N 1/36135; A61N 1/0551; A61N 1/36185

(57) ABSTRACT

A method according to at least one embodiment of the present disclosure includes receiving a first set of data including information about evoked Compound Action Potentials (eCAPs), the first set of data generated by an electrical lead; and training, using the first set of data, an auto-encoder neural network. The auto-encoder neural network may be used in a device with an implantable electrical lead. The auto-encoder neural network may receive information collected by the device, analyze growth curve waveforms, and determine, based on the growth curve waveform analysis, whether or not the implantable electrical lead has moved.

### CROSS-REFERENCE TO RELATED APPLICATIONS

The present application claims the benefit of and priority to U.S. Provisional Application No. 63/338,280 filed on May 4, 2022, entitled “SYSTEMS AND METHODS FOR DETECTING LEAD MOVEMENT”, which application is incorporated herein by reference in its entirety.

### BACKGROUND

The present disclosure is generally directed to neuromodulation and, more specifically, is directed toward detecting lead movement.

The positioning of leads relative to anatomical elements, such as nerves in the spine, can affect patient satisfaction and the amount of relief felt by a patient receiving neuromodulation therapy. The movement of the leads could negatively impact patient outcomes, such as diminished effectiveness of treatment, increased patient pain, or the like.

### BRIEF SUMMARY

Example aspects of the present disclosure include:

A method according to at least one embodiment of the present disclosure comprises: receiving a first set of data including information about evoked Compound Action Potentials (eCAPs), the first set of data generated by an electrical lead; and training, using the first set of data, an auto-encoder neural network.

Any of the features herein, further comprising: receiving a second set of data that is passed into the trained auto-encoder neural network; receiving a set of output data from the trained auto-encoder neural network; and determining, based on the set of output data, whether the electrical lead has moved.

Any of the features herein, wherein the electrical lead is connected to an in-vivo device.

Any of the features herein, wherein the first set of data and the second set of data include data associated with a stimulation amplitude, and wherein the auto-encoder neural network is trained with at least 300 eCAP waveforms.

Any of the features herein, wherein the second set of data includes information describing a stimulated eCAP waveform, wherein the set of output data includes a reconstructed eCAP waveform, and wherein the determining further includes: determining a growth curve loss waveform, the growth curve loss waveform calculated as an absolute value of a difference between the reconstructed eCAP waveform and the stimulated eCAP waveform; classifying, when a mean of the growth curve loss waveform is at or above a threshold value, the electrical lead as moved; and classifying, when the mean of the growth curve loss waveform is below the threshold value, the electrical lead as not moved.

Any of the features herein, wherein the threshold value is a mean of a set of loss values plus a standard deviation of a set of loss values, and wherein each value in the set of loss values is determined based on a difference between each eCAP waveform of the at least 300 eCAP waveforms and a respective reconstructed eCAP waveform generated by the auto-encoder neural network.

Any of the features herein, further comprising: sending, when the electrical lead of the in-vivo device has moved, at least one signal.

Any of the features herein, wherein the signal is configured to alert at least one of an automated programming routine, a manufacturer, an individual, or a physician that the electrical lead of the in-vivo device has moved.

A system according to at least one embodiment of the present disclosure comprises: a processor; and a memory storing data thereon that, when processed by the processor, cause the processor to: receive a first set of information about evoked Compound Action Potentials (eCAPs), the first set of information generated by an electrical lead; and train, using the first set of information, a neural network.

Any of the features herein, wherein the data further cause the processor to: receive a second set of information that is passed into the trained neural network; receive an output from the trained neural network; and determine, based on the output, whether an implanted lead has moved.

Any of the features herein, wherein the first set of information and the second set of information include data about a stimulation amplitude, and wherein the neural network is an auto-encoder neural network trained with at least 300 eCAP waveforms.

Any of the features herein, wherein the second set of information includes a stimulated eCAP waveform, wherein the output includes a reconstructed eCAP waveform, and wherein the data further cause the processor to: determine a growth curve loss waveform, the growth curve loss waveform calculated as an absolute value of a difference between the reconstructed eCAP waveform and the stimulated eCAP waveform; classify, when a mean of the growth curve loss waveform is at or above a threshold value, the implanted lead as moved; and classify, when the mean of the growth curve loss waveform is below the threshold value, the implanted lead as not moved.

Any of the features herein, wherein the threshold value is a mean of a set of loss values plus a standard deviation of a set of loss values, and wherein each value in the set of loss values is determined based on a difference between each eCAP waveform of the at least 300 eCAP waveforms and a respective reconstructed eCAP waveform generated by the neural network.

Any of the features herein, wherein the data further cause the processor to: send, when the implanted lead has moved, at least one signal configured to alert at least one of an automated programming routine, an individual, a manufacturer, or a physician that the implanted lead has moved.

Any of the features herein, wherein the second set of information is captured by an in-vivo device disposed in an individual, and wherein the neural network is unique to the individual.

A device according to at least one embodiment of the present disclosure comprises: a first lead; a processor; and a memory capable of storing data thereon, wherein the data, when processed by the processor, cause the processor to: receive a first set of data including information about evoked Compound Action Potentials (eCAPs), the first set of data capable of being passed into an auto-encoder neural network to train the auto-encoder neural network to detect a movement of the first lead.

Any of the features herein, wherein the data further cause the processor to: train, using the first set of data, the auto-encoder neural network; receive a second set of data that is passed into the auto-encoder neural network; receive an output from the auto-encoder neural network; and determine, based on the output, whether the first lead has moved from a first position to a second position, wherein the first set of data and the second set of data include data associated with a stimulation amplitude, wherein the auto-encoder neural network is trained with at least 300 eCAP waveforms.

Any of the features herein, wherein the second set of data includes a stimulated eCAP waveform, wherein output includes a reconstructed eCAP waveform, and wherein the data further cause the processor to: determine a growth curve loss waveform that is based on an absolute value of a difference between the reconstructed eCAP waveform and the stimulated eCAP waveform; classify, when a mean of the growth curve loss waveform is at or above a threshold value, the first lead as having moved; and classify, when the mean of the growth curve loss waveform is below the threshold value, the first lead as having not moved.

Any of the features herein, wherein the threshold value is a mean of a set of loss values plus a standard deviation of a set of loss values, and wherein each value in the set of loss values is determined based on a difference between each eCAP waveform in the at least 300 eCAP waveforms and a respective reconstructed eCAP waveform generated by the auto-encoder neural network.

Any of the features herein, wherein the data further cause the processor to: generate, when the first lead is classified as having moved, an alert, the alert configured to inform at least one of an automated programming routine, an individual into which the device is disposed, a physician, or a manufacturer that the first lead has moved.

Any feature in combination with any one or more other features.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a diagram of a system according to at least one embodiment of the present disclosure;

FIG. 2A is a diagram of a device with leads connected to nerves according to at least one embodiment of the present disclosure;

FIG. 2B is another diagram of the device with leads connected to nerves according to at least one embodiment of the present disclosure;

FIG. 3 is a diagram of a system according to at least one embodiment of the present disclosure;

FIG. 4 is a diagram of a neural network according to at least one embodiment of the present disclosure;

FIG. 5 is a flowchart according to at least one embodiment of the present disclosure;

FIG. 6 is a flowchart according to at least one embodiment of the present disclosure; and

FIG. 7 shows violin plots depicting loss values associated with leads according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i7 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Spinal cord stimulation lead movement is a leading cause of therapy loss. In some cases, the movement of even 1 millimeter (mm) may largely influence the effectiveness of therapies administered by an in-vivo device. The ability to detect in-vivo lead movements of the leads connected to a device and positioned within (e.g., implanted) a patient could enable the device to automatically notify a physician of a potential lead movement in the patient. The physician may then follow up with a re-programming visit that can restore therapy and improve device usefulness and patient satisfaction. Additionally or alternatively, the detection of in-vivo lead movement may cause the device to generate a signal that activates one or more automated programming routines associated with the in-vivo device. The one or more automated programming routines may cause adjustments to the contacts current is applied through, the amplitude of the current applied to the anatomical element, the frequency, pulse width, charge balance mechanism, current steering, and interleaving of current applied, combinations thereof, and the like. In other words, the one or more automated programming routines may automatically adjust for the lead movement by changing the therapy applied.

The device may be able to sense, measure, and/or record evoked Compound Action Potentials (eCAPs), and the waveforms associated with the eCAPs can be analyzed to detect a change in the waveform that indicates a potential lead movement or migration. In some embodiments, an auto-encoder neural network may analyze the eCAP data to detect lead movement or migration.

In some embodiments, a series of growth curves may be generated after the device and the leads are surgically implanted into the patient. The collected growth curves may be used to train the auto-encoder neural network. In some embodiments, multiple models may be generated to find a steady state of the model as, for example, natural tissue scarring occurs. A population of growth curve loss values may be determined based on the difference between the output of the auto-encoder neural network and the input growth curve. A threshold value may then be determined based on the population of growth curve loss values, such as the mean of the loss values plus the standard deviation of the loss values. The threshold value may be used as a threshold to determine whether a lead has moved (e.g., if an analyzed eCAP waveform has, for example, a mean loss value that is greater than the threshold value, the lead that stimulated the analyzed eCAP waveform may be classified as having moved).

After the lead movement threshold is determined, periodic growth curves may be collected and evaluated with the baseline model to determine a new loss value. In other words, the captured eCAP waveform may be passed through the trained neural network to generate a reconstructed eCAP waveform, and the difference between the reconstructed eCAP waveform and the captured eCAP waveform may be determined (e.g., the absolute value of the difference between the two waveforms) for each data point of the waveform. Based on the set of differences, a new loss value may be determined mathematically, such as a mean of the set. The new loss value may then be compared to the threshold value, and used to predict whether the lead has moved. In some embodiments, the threshold value may be dynamically changed. In other words, the threshold may change from a first threshold value to a different second threshold value based on, for example, additional eCAP waveform data collected.

In the event that the lead is predicted to have moved, a signal, alert, or message may be sent to the patient, physician, and/or the manufacturer for notification and possible intervention. If the lead movement is not detected, the loss values may be used for additional training in the baseline model, to capture additional natural variation.

In some embodiments, the growth curve may be cropped into sections that contain the eCAP waveform, to ensure that data passed into the neural network includes the eCAP waveform. In some embodiments, the neural network may accept stimulation amplitudes and eCAP waveforms, while only eCAP waveforms are output, which enables additional information to be captured in the encoder stage of the neural network to improve prediction performance. In some embodiments, the prediction of the lead moment from the growth curve loss function output may be computed based on a mathematical or statistical manipulation (e.g., a mean) rather than on an individual sample evaluation.

Embodiments of the present disclosure beneficially enable detection of a lead movement of an in-vivo device. Embodiments of the present disclosure also beneficially enable patient, physician, and/or manufacturer intervention for lead movement, reducing the probability that a patient is unsatisfied with the treatment provided by the in-vivo device. Embodiments of the present disclosure further beneficially enable improved troubleshooting of in-vivo devices based on lead movement analysis, allowing the in-vivo device to be adjusted earlier in therapeutic settings and leading to improved patient outcomes and satisfaction.

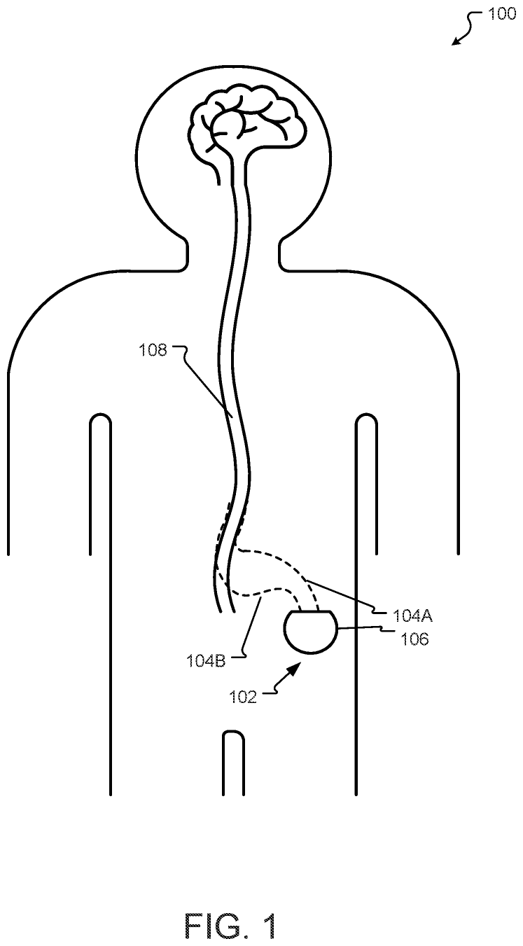

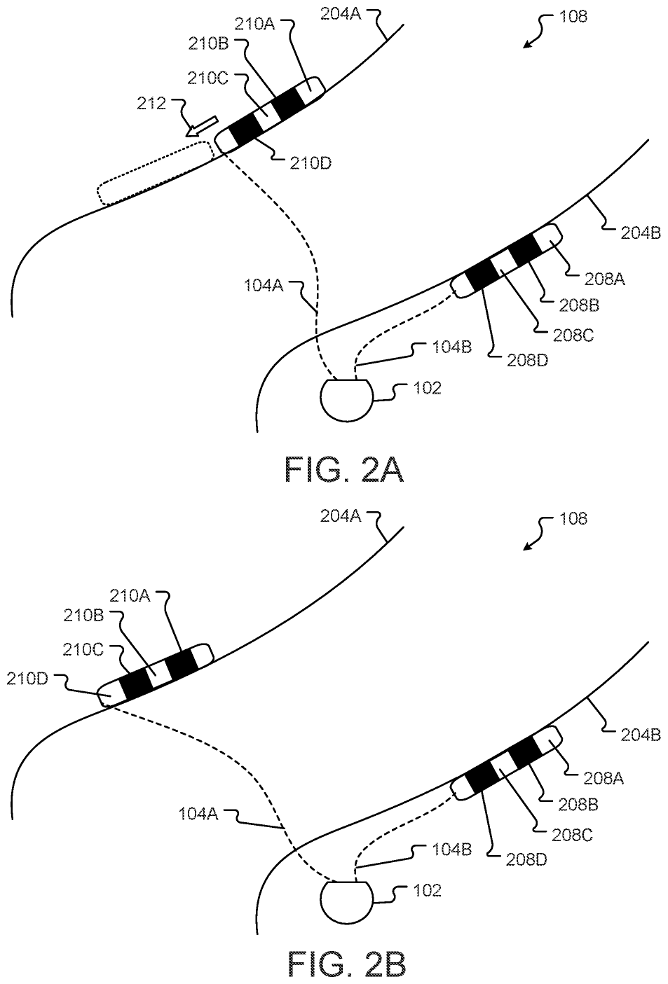

Turning to FIGS. 1-2, diagrams of aspects of a system 100 according to at least one embodiment of the present disclosure is shown. The system 100 may be used to provide electric signals for a patient and/or carry out one or more other aspects of one or more of the methods disclosed herein. For example, the system 100 may include at least a device 102 that is capable of providing a stimulation applied to the spinal cord 108 of the patient and/or to one or more nerve endings for a patient. In some examples, the device 102 may be referred to as an implantable pulse generator 106. More specifically, the implantable pulse generator 106 may be configured to generate a current or electrical signal, such as a signal capable of stimulating an eCAP responses in the spinal cord 108 or from one or more nerves. Additionally, the system 100 may include one or more leads 104 (e.g., electrical leads) that provide a connection between the device 102 and the spinal cord or nerves of the patient for enabling, for example, stimulation. In some embodiments, the leads 104 may be implanted wholly or partially within the patient.

In some embodiments, the one or more leads 104 may include a first lead 104A disposed on or connected to a first side of the spinal cord 108 of the patient and a second lead 104B disposed on or connected to a second side of the spinal cord 108 of the patient. For example, the first lead 104A may be connected to the righthand side of the spinal cord 108, while the second lead 104B may be connected to the lefthand side of the spinal cord 108. However, the position and/or orientation of each lead relative to the spinal cord 108 may vary depending on, for example, the type of treatment, the type of lead, combinations thereof, and the like. In another example, the first lead 104A and the second lead 104B may overlap one another, and may be placed proximate one another on the dorsal side of the spinal cord 108 close to a midline of the spinal cord 108.

In other embodiments, the one or more leads 104 may include at least the first lead 104A and the second lead 104B connected to respective vagal trunks (e.g., different trunks of the vagus nerve) or to other respective nerves in a patient. For example, the first lead 104A may be connected to a first vagal trunk of the patient (e.g., the anterior sub diaphragmatic vagal trunk at the hepatic branching point of the vagus nerve) and the second lead 104B may be connected to a second vagal trunk of the patient (e.g., the posterior sub diaphragmatic vagal trunk at the celiac branching point of the vagus nerve). The first lead 104A and/or the second lead 104B may be configured to provide an electrical stimulation signal from the device 102 to the respective first and/or second vagal trunk. The connection of the leads 104 to the respective vagal trunk (or other nerves) of the patient may permit the device 102 to measure and/or stimulate one or more eCAPs in the patient based on the provided electrical stimulation from the implantable pulse generator 106.

In some examples, the leads 104 may provide the electrical signals to the respective vagal trunks via electrodes or electrode devices that are connected to the vagal trunks (e.g., sutured in place, wrapped around the nerves of the vagal trunks, etc.). In some examples, the leads 104 may be referenced as cuff electrodes or may otherwise include the cuff electrodes (e.g., at an end of the leads 104 not connected or plugged into the device 102).

In other examples, the leads 104 may be or comprise linear spinal cord stimulation (SCS) leads capable of delivering one or more stimulation signals to the spinal cord 108. The leads 104 may comprise a plurality of electrodes disposed along the length of the lead, such that the leads 104 contact the spinal cord 108 at multiple points along a length of the spinal cord 108. A first set of the electrodes on each lead may pass an electrical signal into the spinal cord 108, while a second set of the electrodes on each lead may sense one or more signals generated in response by the spinal cord 108. In one embodiment, the electrodes may be able to sense, measure, or otherwise collected data related to eCAPs (e.g., eCAP waveforms).

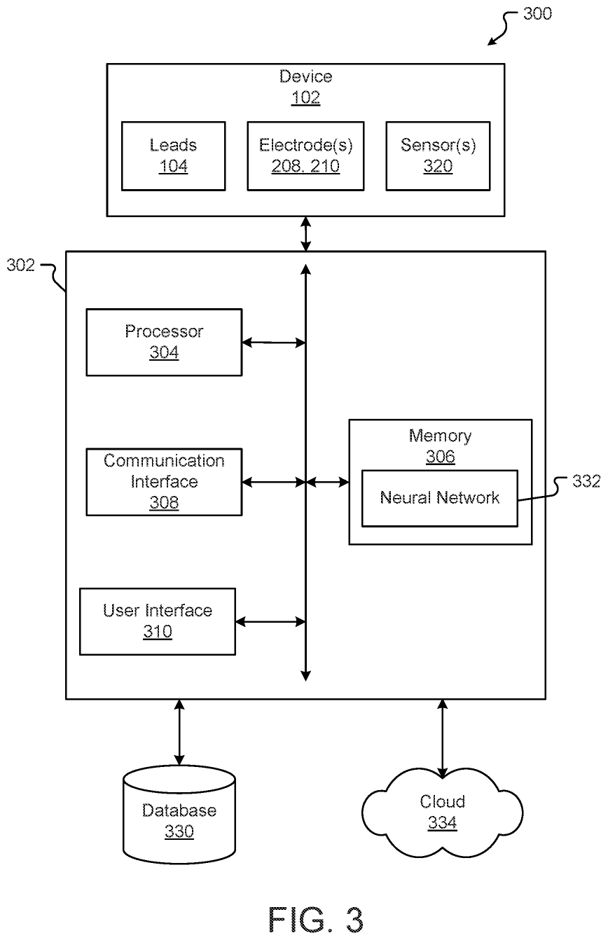

Additionally, while not shown, the system 100 may include one or more processors (e.g., one or more DSPs, general purpose microprocessors, graphics processing units, ASICs, FPGAs, or other equivalent integrated or discrete logic circuitry) shown and described in FIG. 3 that are programmed to carry out one or more aspects of the present disclosure. In some examples, the one or more processors may include a memory or may be otherwise configured to perform the aspects of the present disclosure. For example, the one or more processors may provide instructions to the device 102, the cuff electrodes, or other components of the system 100 not explicitly shown or described with reference to FIG. 1 for stimulating and measuring eCAPs, and analyzing the same, as described herein. In some examples, the one or more processors may be part of the device 102 or part of a control unit for the system 100 (e.g., where the control unit is in communication with the device 102 and/or other components of the system 100).

FIGS. 2A-2B depict the device 102 and the leads 104 connected to the spinal cord 108 of the patient, the leads 104 including one or more electrodes 208, 210 that receive a current or other stimulant instructions from the device 102 (e.g., via the leads 104). In some examples, the electrodes 208, 210 may each include a body and a plurality of electrodes 208A-208D, 210A-210D that are disposed on respective first and second sides 204A, 204B of the spinal cord 108, where the plurality of electrodes 208A-208D, 210A-210D are configured to apply the current generated by the device 102 to the spinal cord 108. As shown, a first electrode 208 may be configured for placement on the spinal cord 108 to apply a current to the spinal cord 108 (e.g., carried via a first lead 104A and emitted from one or more of the electrodes 208A-208D), and a second electrode 210 may also be configured for placement on the spinal cord 108 to apply a current to the spinal cord (e.g., carried via a second lead 104B and emitted from one or more of the electrodes 210A-210D). In some examples, the electrodes 208, 210 may be referred to as cuff electrodes. The application of current to the spinal cord 108 may stimulate an eCAP in the spinal cord or nerve of the patient, and data or information associated with the eCAP may be captured using, for example, one or more sensors, or one or more of the electrodes 208A-208D, 210A-210D. For example, one or more of the electrodes 208A-208D, 210A-210D may generate an electric signal that stimulates the spinal cord 108. The stimulation may cause one or more eCAP responses, which may be sensed, detected, and/or measured by the electrodes 208A-208D, 210A-210D that were not used to stimulate the spinal cord. In some embodiments, the electrodes 208A-208D may stimulate the spinal cord 108, while the electrodes 210A-210D sense the eCAP response. In other embodiments, a first set of electrodes (e.g., the electrodes 208A, 208B, 210A, 210B) may stimulate the spinal cord 108, while a second set of electrodes (e.g., the electrodes 208C, 208D, 210C, 210D) may measure the spinal cord 108 response, including capturing eCAP waveform data.

In some examples, the current being applied to each of the sides 204A, 204B of the spinal cord 108 may be different per electrode 208, 210 or may include different parameters for application to each portion of the spinal cord 108. For example, the first electrode 208 may apply a high frequency stimulation (e.g., such as a given waveform at about 5 kHz) and the second electrode 210 may apply a low frequency stimulation (e.g., such as a square wave or other waveform at 1 Hz) to provide an electrical stimulation signal from the device 102 to the spinal cord 108.

As illustrated in FIG. 2A, the electrode 208 (and by extension the first lead 104A) may shift or move relative to the spinal cord 108, as shown with an arrow 212. The shift may occur naturally, such as when natural tissue scarring displaces the first lead 104A. The movement of the first electrode 208 relative to the spinal cord 108 may result in movement of the first lead 104A, such that the first lead 104A is disposed at a different location of the first side 204A of the spinal cord 108, as shown in FIG. 2B. The movement of the first electrode 208 and/or the first lead 104A relative to the spinal cord 108 (e.g., a movement of 0.2 mm, 0.5 mm, 1 mm, 2 mm, etc.) may alter how electrical signals generated by the device 102 stimulate the spinal cord 108. The difference in stimulation may negatively impact patient treatment. In some embodiments, the movement of the first lead 104A may be reflected in the eCAP waveforms measured from the stimulation of the spinal cord 108 using the first lead 104A. Such differences may be used to determine that the first lead 104A has moved relative to the spinal cord 108, as discussed in further detail below.

The system 100 or similar systems may be used, for example, to carry out one or more aspects of any one of the methods 500 and/or 600 described herein. The system 100 or similar systems may also be used for other purposes. It will be appreciated that the human body has many vagal nerves and the stimulation and/or measurement described herein may be applied to one or more vagal nerves, which may reside at any location of a patient (e.g., lumbar, thoracic, etc.). Further, the use of the leads 104 to stimulate and/or measure eCAPs may occur with different portions of the nervous system. For example, the leads 104 may be connected to one or more of nerve endings in the spinal cord, the brain or portions thereof, combinations thereof, and the like.

Turning to FIG. 3, a block diagram of a system 300 according to at least one embodiment of the present disclosure is shown. The system 300 may be used with the system 100 or components thereof, and/or carry out one or more other aspects of one or more of the methods disclosed herein. The system 300 comprises the device 102, a computing device 302, a database 330, and/or a cloud or other cloud network 334. Systems according to other embodiments of the present disclosure may comprise more or fewer components than the system 300. For example, the system 300 may not include one or more components of the computing device 302, the database 330, and/or the cloud network 334. While the computing device 302 of the system 300 is illustrated as being in communication with the device 102, it is to be understood that the computing device 302 may be disposed as a sub-component within the device 102, or may alternatively be an external device that communicates with the device 102 using, for example, a communication interface 308, or through the cloud 334 or other network. In some embodiments, the device 102 may include any one or more components of the system 300 including, but not limited to, the computing device 302, the processor 304, the memory 306, the communication interface 308, the database 330, the neural network 332, combinations thereof, and the like.

The device 102 may comprise the leads 104, the electrodes 208, 210, and one or more sensors 320. As previously described, the leads 104 and the electrodes 208, 210 may be configured to apply the current to an anatomical element (e.g., the spinal cord, one or more nerves, etc.). The device 102 may communicate with the computing device 302 to receive instructions such as instructions for applying a current to the anatomical element. The device 102 may also provide data (such as data received from the electrodes 208, 210 or measured by the sensors 320), which may be used to optimize the electrodes 208, 210 and/or to optimize parameters of the generated current. The optimization may enable more accurate eCAP waveforms to be stimulated and/or measured (e.g., optimized current may result in less noise in the eCAP waveform signal measured by the sensors 320).

The one or more sensors 320 may be or comprise sensors capable of capturing data and/or information related to eCAP waveforms. For example, the one or more sensors may be or comprise one or more voltmeters or ammeters capable of respectively detecting voltages and currents generated during an eCAP. In some embodiments, the sensors 320 may be directly or proximally attached to the leads 104 to capture data associated with the eCAPs. In some embodiments, the sensors 320 may communicate with the device 102, the computing device 302, and/or the database 330. The communication may enable the sensors 320 to receive instructions from the computing device 302 (e.g., instructions to begin or stop capturing data) and transmit recorded data to, for example, the database 330. In some embodiments, the electrodes 208, 210 may include the one or more sensors 320, or may act as sensors, for measuring the eCAPs.

The computing device 302 comprises a processor 304, a memory 306, a communication interface 308, and a user interface 310. Computing devices according to other embodiments of the present disclosure may comprise more or fewer components than the computing device 302.

The processor 304 of the computing device 302 may be any processor described herein or any similar processor. The processor 304 may be configured to execute instructions stored in the memory 306, which instructions may cause the processor 304 to carry out one or more computing steps utilizing or based on data received from the device 102, the database 330, and/or the cloud network 334. Additionally or alternatively, the processor 304 may be configured to perform tasks, computations, or the like associated with one or more neural networks 332, such as passing data into the one or more neural networks 332, performing one or more transformations on data passed into the one or more neural networks 332, and the like.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, one or more steps of the methods 500 and/or 600 described herein, or of any other methods. The memory 306 may store, for example, instructions and/or machine learning models (e.g., neural networks) that support one or more functions of the device 102. For instance, the memory 306 may store content (e.g., instructions and/or machine learning models) that, when executed by the processor 304, enable lead movement detection.

Content stored in the memory 306, if provided as in instruction, may, in some embodiments, be organized into one or more applications, modules, packages, layers, or engines. Alternatively or additionally, the memory 306 may store other types of content or data (e.g., machine learning models, artificial neural networks, deep neural networks, etc.) that can be processed by the processor 304 to carry out the various method and features described herein. For example, the memory 306 may store the one or more neural networks 332, which may be used to detect and determine lead movement. Thus, although various contents of memory 306 may be described as instructions, it should be appreciated that functionality described herein can be achieved through use of instructions, algorithms, and/or machine learning models. The data, algorithms, and/or instructions may cause the processor 304 to manipulate data stored in the memory 306 and/or received from or via the device 102, the database 330, and/or the cloud network 334.

The computing device 302 may also comprise a communication interface 308. The communication interface 308 may be used for receiving data (for example, data from the device 102) or other information from an external source (such as the device 102, the database 330, the cloud network 334, and/or any other system or component not part of the system 300), and/or for transmitting instructions, images, or other information to an external system or device (e.g., another computing device 302, the device 102, the database 330, the cloud network 334, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 602.11a/b/g/n, Bluetooth, NFC, ZigBee, and so forth). In some embodiments, the communication interface 308 may be useful for enabling the computing device 302 to communicate with one or more other processors 304 or computing devices 302, whether to reduce the time needed to accomplish a computing-intensive task or for any other reason.

The computing device 302 may also comprise one or more user interfaces 310. The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a surgeon or other user to modify instructions to be executed by the processor 304 according to one or more embodiments of the present disclosure, and/or to modify or adjust a setting of other information displayed on the user interface 310 or corresponding thereto.

Although the user interface 310 is shown as part of the computing device 302, in some embodiments, the computing device 302 may utilize a user interface 310 that is housed separately from one or more remaining components of the computing device 302. In some embodiments, the user interface 310 may be located proximate one or more other components of the computing device 302, while in other embodiments, the user interface 310 may be located remotely from one or more other components of the computing device 302.

The database 330 may store information such as patient data, lead parameters, eCAP waveform data or similar data, electrode parameters, neural network weights, threshold values, etc. The database 330 may be configured to provide any such information to the computing device 302 or to any other device of the system 300 or external to the system 300, whether directly or via the cloud network 334. In some embodiments, the database 330 may be or comprise part of a hospital image storage system, such as a picture archiving and communication system (PACS), a health information system (HIS), and/or another system for collecting, storing, managing, and/or transmitting electronic medical records.

The cloud network 334 may be or represent the Internet or any other wide area network. The computing device 302 may be connected to the cloud network 334 via the communication interface 308, using a wired connection, a wireless connection, or both. In some embodiments, the computing device 302 may communicate with the database 330 and/or an external device (e.g., a computing device) via the cloud network 334.

The system 300 or similar systems may be used, for example, to carry out one or more aspects of any of the methods 500 and/or 600 described herein. The system 300 or similar systems may also be used for other purposes.

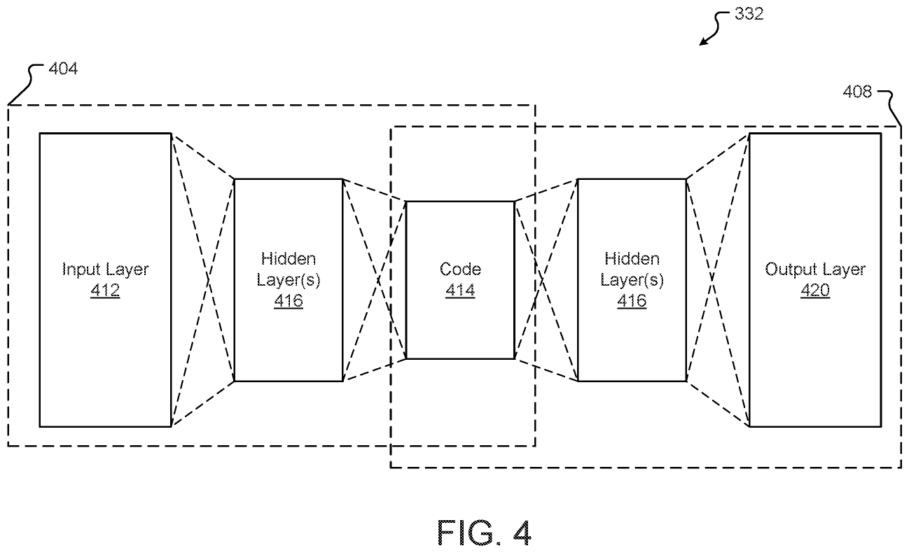

Turning to FIG. 4, aspects of the neural network 332 are shown in accordance with at least one embodiment of the present disclosure. The neural network 332 may be or comprise a collection of nodes and linear or non-linear functions that map one or more inputs to one or more outputs. The neural network 332 may include one or more weights associated with each node that are used with the functions to transform the input into the output as the input passes through the neural network 332. In one embodiment, the neural network 332 includes an encoder 404 and a decoder 408 (e.g., the neural network 332 is an auto encoder neural network). The encoder 404 includes an input layer 412 that receives a discrete or continuous data input, as well as one or more hidden layers 416 through which the data received at the input layer 412 passes. As the data pass through the one or more hidden layers 416, one or more activation functions (e.g., linear transformations, non-linear transformations, etc.) transform the data based on one or more weights. The type of activation function may be, for example, a sigmoid function.

After passing through the one or more hidden layers 416, the transformed data reaches a middle layer, which includes a code 414. The code 414 may be the starting point for the decoder 408 to reconstruct the output. In other words, the code 414 may be a compressed representation of the input that the decoder 408 maps into the output. In some embodiments, the decoder 408 may use similar activation functions to map the information in the code 414 to the output layer 420. The number of layers in the encoder 404 and the decoder 408, the number of nodes in each layer, the size of the code 414 (e.g., the number of nodes in the middle layer), the dimensionality of the data, the type and number of activation functions, and the like are not particularly limited, and may vary based on embodiment.

The training the of the neural network 332 may include passing sets of training data into the encoder 404 and receiving a reconstruction from the decoder 408. In some embodiments, the training data may be or comprise eCAP waveforms, and may include information associated with the shape of the eCAP waveform and/or the stimulation amplitude used in generating the eCAP waveform. Additionally or alternatively, other measurement information may be passed into the neural network 332 (e.g., data measured by one or more accelerometers disposed in the device 102). A loss function defined by error between the initial data and the reconstruction may be calculated therefrom, and used to adjust the weights in the neural network 332. In some embodiments, the error may be determined based on a mean squared error (e.g., the difference between the reconstruction and the initial data at each value is determined, squared, summed together, and divided by the number of data points). The use of mean squared error as the loss function is not limiting, however, and alternative loss functions could be used to determine the error, such as a loss function based on cross entropy. In some embodiments, the mean squared error for each training set may be saved and stored in the database 330. In other words, after the neural network 332 outputs a reconstruction of a set of input data (e.g., an eCAP waveform) and a mean squared error (or other error measure) is determined, the mean squared error value may be saved to the database 330. This process of saving the mean squared error value may occur for some or all of the input eCAP waveforms (e.g., for some or all of the training data), with the resulting set of means squared error data forming a separate distribution that may be used, for example, to define a threshold used to determine lead movement.

Once the loss function has been determined, the loss function data may be backpropagated through the neural network 332 from the decoder 408 to the encoder 404. During the backpropagation, the contribution of each weight of each node in each layer to the overall error may be determined and adjusted based on an optimization method (e.g., gradient descent, stochastic gradient descent, etc.), resulting in a change in the value of some or all of the weights. Stated differently, the error may be used to optimize the weights of the neural network 332 to reduce the error associated with subsequent use of the neural network 332. In some embodiments, the training may be based on a fixed amount of training data (e.g., 2000 data sets), while in other embodiments the training may last until the error between the reconstruction and the input data fall within a range of values or below a threshold value. In some embodiments, the neural network 332 may be continuously trained in conjunction with use. For example, data sets that have minimal error may be backpropagated even if the data set is not part of the training set to better capture, for example, minor shifts in lead positioning as tissues scars around the lead.

In some embodiments, the neural network 332 may be trained on data unique to an individual (e.g., eCAPs generated by nerves of an individual), and may be used only on the specific individual. In other words, the training data used to train the neural network 332 may be collected by the in-vivo device implanted in the individual, beneficially enabling the lead movement detection to be individualized to the individual. By using training sets unique to the individual, variance in variables like frequency and magnitude of eCAPs that may naturally vary across a population of individuals can be reduced or mitigated, resulting in fewer false positives when classifying a lead as having moved.

The neural network 332 or similar neural networks may be used, for example, to carry out one or more aspects of the methods 500 and/or 600 described herein. The neural network 332 or similar neural networks may also be used for other purposes.

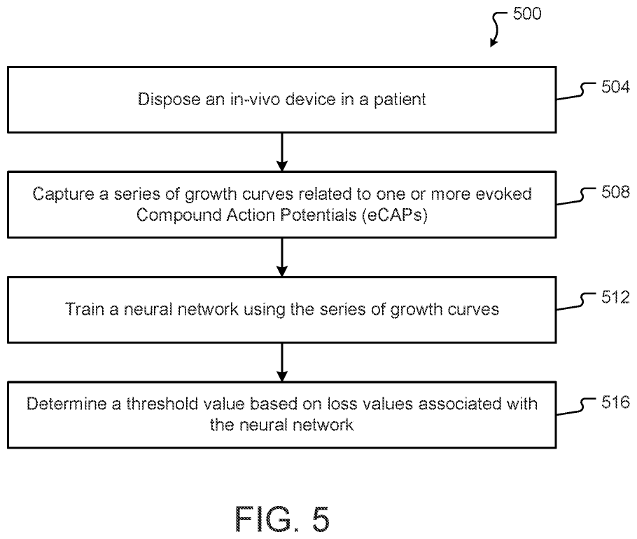

FIG. 5 depicts a method 500 that may be used, for example, to implant a device and train a neural network based on measurements made by the device.

One or more steps of the method 500 may be carried out or otherwise performed, for example, by at least one processor. The at least one processor may be the same as or similar to the processor(s) 304 of the computing device 302 described above. The at least one processor may be part of a device (such as a device 102). A processor other than any processor described herein may also be used to execute the method 500. The at least one processor may perform steps of the method 500 by executing elements stored in a memory such as the memory 306. The elements stored in the memory and executed by the processor may cause the processor to execute one or more steps of a function as shown in method 500. One or more portions of a method 500 may be performed by the processor executing any of the contents of memory, such as a neural network 332.

The method 500 comprises disposing an in-vivo device in a patient (step 504). In some embodiments, the in-vivo device may be similar to or the same as the device 102, and may include leads 104, with the leads 104 also implanted in the patient. The device 102 may include an implantable pulse generator 106 capable of receiving instructions to stimulate the nerves or environment connected to or coupled with the leads 104. In some embodiments, the device 102 may be implanted such that a first lead 104A of the device 102 connects with the spinal cord 108 of the patient. The device 102 may be disposed partially or entirely within the patient, and may be accessible such that the first lead 104A and/or the second lead 104B can be repositioned, reconnected, or otherwise be manipulated by a user (e.g., by a physician).

The method 500 also comprises capturing a series of growth curves related to one or more eCAPs (step 508). The eCAPs may be stimulated by passing a current through the first lead 104A and/or the second lead 104B and into one or more nerves. In some embodiments, the eCAPs may be or comprise naturally-occurring eCAPs or, in other words, eCAPs that are not created by stimulating one or more nerves using the device 102 or components thereof. The eCAPs may be captured by the device 102 using, for example, one or more sensors 320, and may be stored in a database 330.

In some embodiments, the measured eCAPs may be filtered or otherwise transformed (e.g., filtered through a band-pass filter, filtered through a low-pass filter, etc.) before being saved or stored in the database 330. The filtering may be performed to ensure that an eCAP waveform is present in the captured data, or may be performed for any other reason. Stated differently, some data captured from the growth curve may not depict the eCAP waveform (e.g., the eCAP waveform may be a portion of the overall growth curve), and the filtering and/or transformations may be used to ensure that the eCAP waveform is saved or stored. The saved eCAP waveform may then be used, for example, in training a neural network to detect eCAP waveforms. The measured eCAP data may include information associated with a stimulation amplitude used in generating the eCAP waveform (e.g., a magnitude of the stimulation used to trigger the eCAP response), or may otherwise include information from which the stimulation amplitude can be derived.

The method 500 also comprises training a neural network using the series of growth curves (step 512). The neural network may be similar to or the same as the neural network 332. In other words, the neural network may include an encoder 404 and a decoder 408 that are both trained to detect eCAPs based on the provided training data. In some embodiments, the growth curves (and/or the portions of the growth curves containing eCAP waveforms) may be input into the encoder 404, and a reconstruction may be generated as an output of the decoder 408. The reconstruction may then be compared to the initial input data. Based on the comparison, the step 512 may backpropagate a measurement of the error (e.g., the mean squared error) through the neural network 332, and may use an optimization method (e.g., gradient descent, stochastic gradient descent, etc.) to adjust the weights associated with the nodes in the neural network. In one embodiment, the neural network 332 may be trained on data that includes information about the stimulation amplitude and the eCAP waveform. In this embodiment, the stimulation amplitude (e.g., the magnitude of stimulation generated by the device 102) and the eCAP waveform may be concatenated together (e.g., the eCAP waveform data and the stimulation amplitude may be stored in a single matrix) to form a set of data that is used to train the neural network. The use of the stimulation amplitude with the eCAP waveform as an input may reduce or mitigate the influence of variance in eCAP waveforms based on, for example, circadian rhythms.

In some embodiments, the neural network 332 may be trained on one or more sets of data containing eCAP waveforms collected over a period of time (e.g., eCAPs recorded over 24 hours, 48 hours, 72 hours, 7 days, 10 days, 14 days, 21 days, etc.). The period of time may permit for natural movement of the in-vivo device and/or the leads toward a steady state, such as when natural tissue scarring develops. The period of time, however, is not particularly limited, and eCAP waveform data collected from any timeframe may be used to train the neural network 332. Further, the number of eCAP waveforms contained in the data set used to train the neural network is not particularly limited, and any number of eCAP waveforms may be used to train the neural network. Non-limiting examples include 300, 500, 600, 800, 1000, 1500, 2000, 2500, 5000, and 10000 eCAP waveforms.

The method 500 also comprises determining a threshold value based on loss values associated with the neural network (step 516). As previously noted, one or more eCAP waveforms may be used to train the neural network. After each reconstruction is output from the decoder 408 during training, the mean squared error between the reconstruction and the input eCAP waveform may be determined and stored in the database 330. Over the course of training the neural network, the collection of individual loss values (e.g., values of the mean squared error for each reconstruction of each eCAP waveform) form a separate loss value distribution. The loss value distribution may be used to create a threshold value, with the threshold value used as a basis for determining whether a lead has moved, as described below in the method 600. In some embodiments, the threshold value may be based on performing one or more mathematical or statistical calculations based on the loss value distribution. For example, the threshold value may be set as the mean of the loss value distribution plus the standard deviation of the loss value distribution. In another example, the threshold value may be set as the mean of the loss value distribution plus three times the standard deviation of the loss value distribution. In another example, the threshold value may be set as the median of the loss value distribution plus 0.25 times the range of the loss value distribution. In yet another example, the threshold value may be based on a p-value (e.g., a value reflective of the probability that the difference in eCAP waveforms distributions occurred by chance) and a calculated t-value (e.g., based on a paired t-test, an unpaired t-test, etc.) based on the eCAP waveform data. In this example, the p-value may be used to determine a t-table value (e.g., a value based on the degrees of freedom and the p-value of a normally distributed eCAP waveform), with the t-table value compared to the calculated t-value to determine whether the eCAP waveform is significant. If the t-value is greater than the t-table value, the eCAP waveform may be classified as significant (e.g., the lead has moved), while when the t-value is lesser than the t-table value, the eCAP waveform may be classified as non-significant (e.g., the lead has not moved). Notwithstanding the foregoing examples, the present disclosure is in no way limiting to the formulation of the threshold value based on the loss value distribution, and alternative threshold value definitions are possible.

The present disclosure encompasses embodiments of the method 500 that comprise more or fewer steps than those described above, and/or one or more steps that are different than the steps described above.

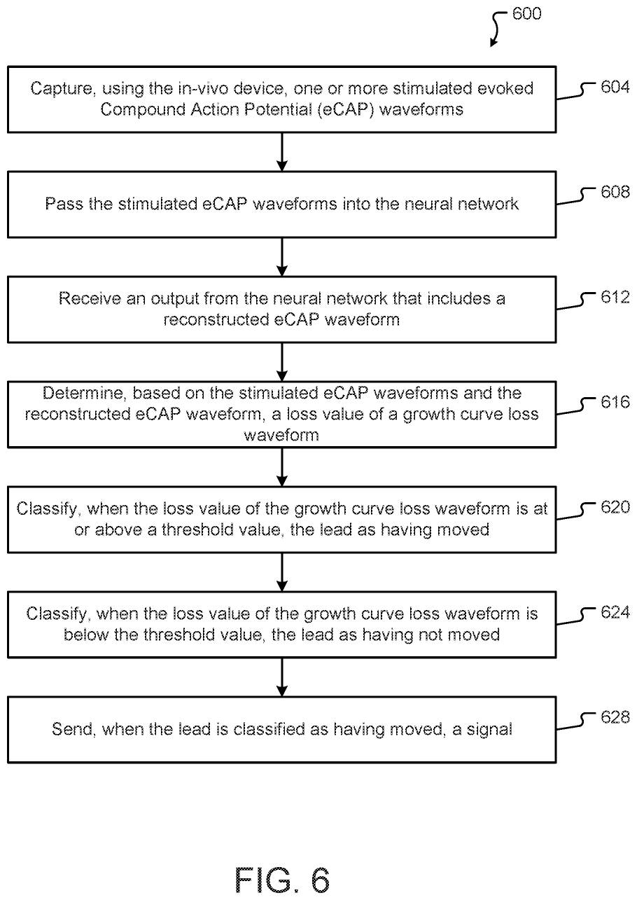

FIG. 6 depicts a method 600 that may be used, for example, to determine whether a lead has moved.

The method 600 (and/or one or more steps thereof) may be carried out or otherwise performed, for example, by at least one processor. The at least one processor may be the same as or similar to the processor(s) 304 of the computing device 302 described above. The at least one processor may be part of a device (such as a device 102). A processor other than any processor described herein may also be used to execute the method 600. The at least one processor may perform the method 600 by executing elements stored in a memory such as the memory 306. The elements stored in the memory and executed by the processor may cause the processor to execute one or more steps of a function as shown in method 600. One or more portions of a method 600 may be performed by the processor executing any of the contents of memory, such as a neural network 332.

The method 600 comprises capturing, using an in-vivo device, one or more stimulated eCAP waveforms (step 604). The one or more stimulated eCAP waveforms may be generated based on eCAPs stimulated by the implantable pulse generator 106. For example, the processor 304 may instruct the implantable pulse generator 106 to generate a current in one or more nerves to which the device 102 is connected. The current may stimulate an eCAP response in one or more nerves. The resulting eCAP waveform may be captured (e.g., using one or more sensors 320) and stored in the database 330. In some embodiments, the stimulation amplitude (e.g., the magnitude of the stimulation used to generate the eCAP response) may also be recorded and stored in the database 330.

The method 600 also comprises passing the stimulated eCAP waveforms into the neural network (step 608). In some embodiments, the neural network may be similar to or the same as the neural network 332. In some embodiments, the neural network 332 may be trained on eCAP waveform and stimulation amplitude data, such as in the step 512 of the method 500. The stimulated eCAP waveforms may be passed into an input layer 412 of the neural network 332. In some embodiments, the stimulation amplitude may be concatenated with the eCAP waveform data, and the concatenated data may be passed into the input layer 412 of the neural network 332.

The method 600 also comprises receiving an output from the neural network that includes a reconstructed eCAP waveform (step 612). The one or more stimulated eCAP waveforms may pass through the hidden layers 416, the code 414, and exit the neural network 332 through the output layer 420. The output data from the output layer 420 may include a separate reconstructed eCAP waveform for each of the one or more stimulated eCAP waveforms passed into the neural network 332.

The method 600 also comprises determining, based on the stimulated eCAP waveform and the reconstructed eCAP waveform, a growth curve loss waveform (step 616). The stimulated eCAP waveform may be the waveform captured by the device 102, while the reconstructed eCAP waveform may be the eCAP waveform predicted by the neural network 332. The growth curve loss waveform may be determined by calculating an absolute value of the difference between the stimulated eCAP waveform and the reconstructed eCAP waveforms. In other words, each point in the curve loss waveform may be the error associated with the difference between the stimulated eCAP waveform and the reconstructed eCAP waveform at the point. Based on the collection of error values, a loss value associated with the growth curve loss waveform may be determined. The loss value may be one or more mathematical or statistical measurements of the growth curve loss waveform, and may be or represent an overall measure of the error of the growth curve loss waveform. For example, the loss value may be the mean of the growth curve loss waveform. As another example, the loss value may be the median of the growth curve loss waveform. However, the above examples are in no way limiting, and additional or alternative mathematical or statistical measurements or combinations thereof may be used to determine the loss value.

The method 600 also comprises classifying, when the loss value of the growth curve loss waveform is at or above a threshold value, the lead as having moved (step 620). The loss value determined based on, for example, the mean of the growth curve loss waveform. In some embodiments, the threshold value may be a threshold value determined in the step 516 of the method 500. For example, the threshold value may be or comprise a t-table value (which may be derived based on a p-value), while a t-value may be determined based on the data related to the growth curve loss waveform. The classification may then be based on whether the t-value is greater than or less than the t-table value: when the t-value is greater than the t-table value, the lead may be classified has having moved, while a t-value lesser than the t-table value may result in the lead being classified as having not moved. The comparison may determine whether the loss value of the growth curve equals or is greater than the threshold value. The loss value of the growth curve being equal to or exceeding the threshold value may indicate that the stimulated waveform passed into the neural network 332 was generated by the lead when the lead was in a different position than the lead was when the neural network 332 was trained. In other words, the loss value of the growth curve may be significant enough that the neural network 332 created a poor reconstruction of the eCAP waveform, which may indicate that the lead has moved. In some embodiments, the step 620 may cause a value associated with a lead position in the database 330 to be updated (e.g., a binary value associated with the lead may be updated from a value of zero to a value of one) to reflect a classification of having moved.

The method 600 also comprises classifying, when the loss value of the growth curve loss waveform is below a threshold value, the lead as having not moved (step 624). In the event that the loss value of the growth curve loss waveform falls below the threshold value, the lead may be classified as having not moved. In some embodiments, when the loss value falls below the threshold value, the error associated with the collected eCAP waveform may be backpropagated to further train the neural network 332. In such embodiments, the neural network 332 can continue to be trained as, for example, the lead position adapts to natural tissue scarring.

In some embodiments, any one or more of the previous steps of the method 600 may be performed for one or more leads connected to the device 102. For example, the steps 604 through 628 may be performed for the first lead 104A, and may then be repeated for the second lead 104B. As a result, different neural networks may be trained based on each individual lead, and each individual lead may be subject to a movement analysis. In other embodiments, a single neural network may be trained using eCAP waveform data generated from both leads and used for the classification of each individual lead as having moved or having not moved.

The method 600 also comprising sending, when the lead is classified as having moved, a signal (step 628). The signal may be generated by the device 102 and/or the computing device 302. The signal may indicate that the lead being classified has been classified as having moved. In some embodiments, the signal may be an alert sent to the patient (e.g., the user into which the in-vivo device has been implanted), a physician, and/or a manufacturer. The alert may indicate that the lead has moved. The alert may generate a visual, auditory, or kinesthetic signal. For example, the signal may create a message alert on a user device (e.g., a mobile phone or other user device), may generate an automated phone message, may generate an auditory tone (e.g. a siren or other alarm), may cause a user device to vibrate, combinations thereof, and the like to alert the recipient of the signal that the lead has moved. In some embodiments, a separate signal may be generated for each lead of the in-vivo device. In such embodiments, the separate signals may be unique to the lead, and may contain additional information about the lead (e.g., the signal may indicate which lead has moved, when the lead move was detected, etc.). In some embodiments, the alert may activate one or more automated programming routines associated with the in-vivo device. The one or more automated programming routines may cause adjustments to the contacts used to supply the current, the amplitude of the current applied to the anatomical element, the frequency, pulse width, current steering, and interleaving of current applied, combinations thereof, and the like.

The signal may beneficially enable the moved lead to be adjusted or addressed to improve patient treatment and outcomes. For example, in some instances a lead may be classified as having moved, and an alert may be generated and sent to the patient's physician. The physician may then reach out to the patient to verify patient satisfaction. In the event that the patient has seen a degradation in treatment experience, the physician can schedule a follow-up appointment to adjust the device. As a result, patient outcomes and satisfaction can be improved with embodiments of the present disclosure.

The present disclosure encompasses embodiments of the method 600 that comprise more or fewer steps than those described above, and/or one or more steps that are different than the steps described above.

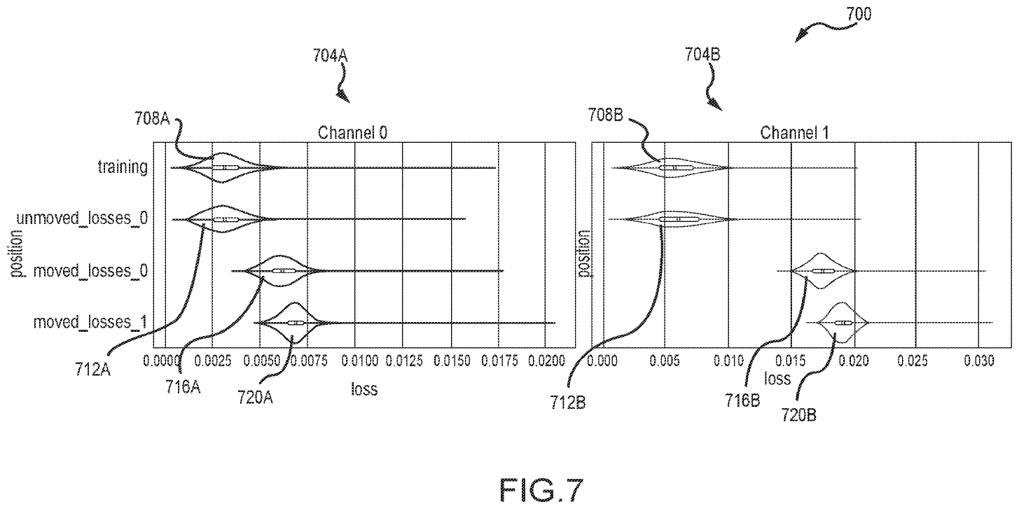

FIG. 7 depicts violin plots 700 of loss values in accordance with at least one embodiment of the present disclosure. The violin plots 700 may be generated based on the methods 500 and 600, and include a first violin plot 704A and a second violin plot 704B. In some embodiments, the first violin plot 704A may correspond to data collected from stimulations created by the first lead 104A, while the second violin plot 704B may correspond to data collected from stimulations created by the second lead 104B. The loss values may be the difference between the reconstructed eCAP waveforms and the stimulated eCAP waveforms.

The training sets 708A-708B illustrate loss values associated with the training data. The violin plots 700 may also reflect the distribution of loss values for when the lead has moved and not moved. The unmoved lead plots 712A-712B depict loss values associated with eCAP waveforms from a lead that has not moved. The first moved lead plots 716A-716B depict distributions of loss values when the respective lead has been moved to a first position. Similarly, the second moved lead plots 720A-720B depict distributions of loss values when the respective lead has been moved to a second position. As can be seen in the first violin plot 704A and the second violin plot 704B, the unmoved lead plots 712A-712B are closer in distribution, mean, and range to the respective training sets 708A-708B than the first moved lead plots 716A-716B and the second moved lead plots 720A-720. Stated differently, the first moved lead plots 716A-716B and the second moved lead plots 720A-720B illustrate distributions that, on average, contain larger loss values than the training sets 708A-708B and the unmoved lead plots 712A-712. The larger loss values may indicate that the neural network used to generate the reconstructed eCAP waveforms performed worse for the moved lead than for the unmoved lead.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method, comprising:
receiving a first set of data including information about evoked Compound Action Potentials (eCAPs), the first set of data generated by an electrical lead; and
training, using the first set of data, an auto-encoder neural network.

2. The method of claim 1, further comprising:
receiving a second set of data that is passed into the trained auto-encoder neural network;
receiving a set of output data from the trained auto-encoder neural network; and
determining, based on the set of output data, whether the electrical lead has moved.

3. The method of claim 2, wherein the electrical lead is connected to an in-vivo device.

4. The method of claim 3, wherein the first set of data and the second set of data include data associated with a stimulation amplitude, and wherein the auto-encoder neural network is trained with at least 300 eCAP waveforms.

5. The method of claim 4, wherein the second set of data includes information describing a stimulated eCAP waveform, wherein the set of output data includes a reconstructed eCAP waveform, and wherein the determining further includes:
determining a growth curve loss waveform, the growth curve loss waveform calculated as an absolute value of a difference between the reconstructed eCAP waveform and the stimulated eCAP waveform;
classifying, when a mean of the growth curve loss waveform is at or above a threshold value, the electrical lead as moved; and
classifying, when the mean of the growth curve loss waveform is below the threshold value, the electrical lead as not moved.

6. The method of claim 5, wherein the threshold value is a mean of a set of loss values plus a standard deviation of a set of loss values, and wherein each value in the set of loss values is determined based on a difference between each eCAP waveform of the at least 300 eCAP waveforms and a respective reconstructed eCAP waveform generated by the auto-encoder neural network.

7. The method of claim 5, further comprising:
sending, when the electrical lead of the in-vivo device has moved, at least one signal.

8. The method of claim 7, wherein the signal is configured to alert at least one of an automated programming routine, a manufacturer, an individual, or a physician that the electrical lead of the in-vivo device has moved.

9. A system, comprising:
a processor; and
a memory storing data thereon that, when processed by the processor, cause the processor to:
receive a first set of information about evoked Compound Action Potentials (eCAPs), the first set of information generated by an electrical lead; and
train, using the first set of information, a neural network.

10. The system of claim 9, wherein the data further cause the processor to:
receive a second set of information that is passed into the trained neural network;
receive an output from the trained neural network; and
determine, based on the output, whether an implanted lead has moved.

11. The system of claim 10, wherein the first set of information and the second set of information include data about a stimulation amplitude, and wherein the neural network is an auto-encoder neural network trained with at least 300 eCAP waveforms.

12. The system of claim 11, wherein the second set of information includes a stimulated eCAP waveform, wherein the output includes a reconstructed eCAP waveform, and wherein the data further cause the processor to:
determine a growth curve loss waveform, the growth curve loss waveform calculated as an absolute value of a difference between the reconstructed eCAP waveform and the stimulated eCAP waveform;
classify, when a mean of the growth curve loss waveform is at or above a threshold value, the implanted lead as moved; and
classify, when the mean of the growth curve loss waveform is below the threshold value, the implanted lead as not moved.

13. The system of claim 12, wherein the threshold value is a mean of a set of loss values plus a standard deviation of a set of loss values, and wherein each value in the set of loss values is determined based on a difference between each eCAP waveform of the at least 300 eCAP waveforms and a respective reconstructed eCAP waveform generated by the neural network.

14. The system of claim 12, wherein the data further cause the processor to:
send, when the implanted lead has moved, at least one signal configured to alert at least one of an automated programming routine, an individual, a manufacturer, or a physician that the implanted lead has moved.

15. The system of claim 12, wherein the second set of information is captured by an in-vivo device disposed in an individual, and wherein the neural network is unique to the individual.

16. A device, comprising:
a first lead;
a processor; and
a memory capable of storing data thereon, wherein the data, when processed by the processor, cause the processor to:
receive a first set of data including information about evoked Compound Action Potentials (eCAPs), the first set of data capable of being passed into an auto-encoder neural network to train the auto-encoder neural network to detect a movement of the first lead.

17. The device of claim 16, wherein the data further cause the processor to:
train, using the first set of data, the auto-encoder neural network;
receive a second set of data that is passed into the auto-encoder neural network;
receive an output from the auto-encoder neural network; and
determine, based on the output, whether the first lead has moved from a first position to a second position, wherein the first set of data and the second set of data include data associated with a stimulation amplitude, wherein the auto-encoder neural network is trained with at least 300 eCAP waveforms.

18. The device of claim 17, wherein the second set of data includes a stimulated eCAP waveform, wherein output includes a reconstructed eCAP waveform, and wherein the data further cause the processor to:
determine a growth curve loss waveform that is based on an absolute value of a difference between the reconstructed eCAP waveform and the stimulated eCAP waveform;
classify, when a mean of the growth curve loss waveform is at or above a threshold value, the first lead as having moved; and
classify, when the mean of the growth curve loss waveform is below the threshold value, the first lead as having not moved.

19. The device of claim 18, wherein the threshold value is a mean of a set of loss values plus a standard deviation of a set of loss values, and wherein each value in the set of loss values is determined based on a difference between each eCAP waveform in the at least 300 eCAP waveforms and a respective reconstructed eCAP waveform generated by the auto-encoder neural network.

20. The device of claim 19, wherein the data further cause the processor to:
Generate, when the first lead is classified as having moved, an alert, the alert configured to inform at least one of an automated programming routine, an individual into which the device is disposed, a physician, or a manufacturer that the first lead has moved.
